(19) United States
(12) Patent Application Publication
(10) Pub. No.: US 2011/0136151 A1
(43) Pub. Date: Jun. 9, 2011

(54) ASSAYS FOR DIAGNOSING AND EVALUATING TREATMENT OPTIONS FOR POMPE DISEASE

(75) Inventors: Brandon Wustman, San Diego, CA (US); Hung V. Do, New Hope, PA (US)

(73) Assignee: AMICUS THERAPEUTICS, INC., Cranbury, NJ (US)

(21) Appl. No.: 12/920,856

(22) PCT Filed: Mar. 12, 2009
(86) PCT No.: PCT/US09/36989

§ 371 Date: Feb. 25, 2011

Related U.S. Application Data
(60) Provisional application No. 61/035,866, filed on Mar. 12, 2008.

(51) Int. Cl. G01N 33/53

(52) U.S. Cl. 435/7.24; 435/7.21

(57) ABSTRACT

Provided are in vitro, ex vivo and in vivo methods for determining whether a patient with Pompe disease will respond to treatment with a specific pharmacological chaperone.

### CROSS-REFERENCE TO RELATED APPLICATIONS

This application claims priority to U.S. Provisional Application No. 61/035,866 filed Mar. 12, 2008; the contents of which are incorporated herein by reference.

### FIELD OF THE INVENTION

The present invention provides methods to determine whether a patient with Pompe disease will benefit from treatment with a specific pharmacological chaperone. The present invention exemplifies several cell-based in vitro, ex vivo and in vivo methods for determining the responsiveness of acid α-glucosidase (GAA) variants to a pharmacological chaperone such as 1-deoxynojirimycin (DNJ). An in situ application of the method also provides a way to identify Pompe patients and obtain useful information on dosing these pharmacological chaperones. A novel method to accurately measure GAA activity in tissue homogenate samples is also a subject of the present invention.

### BACKGROUND

Pompe disease is an inherited metabolic disorder that is one of approximately forty lysosomal storage disorders (LSDs). These LSDs are a group of autosomal recessive diseases caused by the accumulation of cellular glycosphingolipids, glycogen, or mucopolysaccharides, due to defective hydrolytic enzymes. Examples of lysosomal disorders include but are not limited to Gaucher disease (Beutler et al., The Metabolic and Molecular Bases of Inherited Disease. 8th ed. 2001 Scriver et al., ed. pp. 3635-3668, McGraw-Hill, New York), G_{M1}-gangliosidosis (id. at pp 3775-3810), fucosidosis (The Metabolic and Molecular Bases of Inherited Disease 1995. Scriver, C. R., Beaudet, A. L., Sly, W. S. and Valle, D., ed pp. 2529-2561, McGraw-Hill, New York), mucopolysaccharidoses (id. at pp 3421-3452), Pompe disease (id. at pp. 3389-3420), Hurler-Scheie disease (Weismann et al., Science. 1970; 169, 72-74), Niemann-Pick A and B diseases, (The Metabolic and Molecular Bases of Inherited Disease 8th ed. 2001. Scriver et al. Ed. pp 3589-3610, McGraw-Hill, New York), and Fabry disease (Id. at pp. 3733-3774).

The specific pharmacological chaperone (“SPC”) strategy has been demonstrated for numerous enzymes involved in lysosomal storage disorders as in U.S. Pat. Nos. 6,274,597, 6,583,158, 6,589,964, 6,599,919, and 6,916,829 to Fan et al., which are incorporated herein by reference in their entirety. For example, a small molecule derivative of galactose, 1-deoxygalactonojirimycin (DGJ), a potent competitive inhibitor of the mutant Fabry enzyme α-galactosidase A (α-Gal A: GLA), effectively increased in vitro stability of the human mutant α-Gal A (R301Q) at neutral pH, and it enhanced the mutant enzyme activity in lymphoblasts established from Fabry patients with R301Q or Q279E mutations. Furthermore, oral administration of DGJ to transgenic mice overexpressing mutant (R301Q) α-Gal A substantially elevated the enzyme activity in major organs (Fan et al. Nature Med. 1999; 5: 112-115). Similar rescue of glucocerebrosidase (acid β-glucosidase, GBA) from Gaucher patient cells has been described using another iminosugar, isofagomine (IFG), and its derivatives, described in U.S. Pat. No. 6,916,829, and using other compounds specific for glucocerebrosidase (described in pending U.S. patent application Ser. Nos. 10/988,428, and 10/988,427, both filed Nov. 12, 2004). U.S. Pat. No. 6,583,158, described above, discloses several small molecule compounds that would be expected to stabilize mutant GAAs and increase cellular levels of the enzyme for the treatment of Pompe disease, including 1-deoxynojirimycin (DNJ), α-homonojirimycin, and castanospermine.

However, as indicated above, successful candidates for SPC therapy must have a mutation which results in the production of an enzyme that has the potential to be stabilized and folded into a conformation that permits trafficking out of the ER. Mutations which severely truncate the enzyme, such as nonsense mutations, or mutations within the catalytic domain which prevent binding of the chaperone, will not likely be “rescuable” or “enhanceable” using SPC therapy. However, it is often difficult to predict responsiveness of specific mutations even if they are outside the catalytic site and requires empirical experimentation. Moreover, since WBCs only survive for a short period of time in culture (ex vivo), screening for SPC enhancement of GAA is difficult.

In order to apply SPC therapy effectively, a broadly applicable, fast and efficient method for screening patients for responsiveness to SPC therapy needs to be adopted prior to initiation of treatment. Thus, there remains in the art a need for relatively non-invasive methods to rapidly assess the potential for enzyme enhancement via SPCs prior to making treatment decisions, for both cost and emotional benefits to the patient.

### SUMMARY OF THE INVENTION

The present invention provides in vitro and ex vivo assays to evaluate GAA activity in a model mammalian expression system and freshly-isolated lymphocytes derived from patients with Pompe disease in the presence or absence of a SPC, in order to determine whether a patient is a candidate for SPC therapy and, optionally, to evaluate the extent of successful treatment. The present invention also includes the basis for evaluation of SPC as a treatment option for other protein abnormalities and/or enzyme deficiencies (e.g. protein deficiencies resulting from cystic fibrosis. α-1-antitrypsin deficiency, familial hypercholesterolemia. Fabry disease, and Alzheimer's disease. For additional protein deficiencies, see U.S. patent application publication 20060153829, herein incorporated by reference in its entirety.).

One aspect of the present application, relates to an improved method of diagnosing Pompe disease by determining, GAA activity in isolated leukocytes (e.g. T cells) from patients suspected of having Pompe disease.

A second aspect of the present application provides an improved method of diagnosing Pompe disease by determining GAA activity in lymphoblast and/or fibroblast cell lines derived from patients suspected of having Pompe disease.

The present invention also provides methods of measuring GAA enzyme activity in situ in freshly isolated leukocytes to evaluate the response of GAA to SPC therapy and information about the effectiveness of various dosing regimens. For example, the present application further provides methods for evaluating, an in vivo GAA response to SPC therapy after a treatment period.

The present invention also provides diagnostic kits containing the components required to perform assays of the present application.

The present invention further provides a method to accurately measure GAA activity in Tissue homogenate samples.

### BRIEF DESCRIPTION OF THE DRAWINGS

FIG. 1. Shows the effect of DNJ on patient-derived lymphoblasts isolated from Pompe disease patients with different mutations in their α-glucosidase (GAA) enzyme.

### DETAILED DESCRIPTION

The present invention provides several assays to allow the accurate determination of whether an SPC enhances enzyme activity from cells derived from patients with Pompe disease. These assays permit a determination of whether the patient will be a candidate for SPC therapy.

The new ex vivo assay is sufficiently sensitive and can be performed on freshly isolated leukocytes to obtain pertinent information on the whether a patient is amenable to SPCs. This assay utilizes various substrates (e.g., fluorogenic substrates known in the art, natural glycogen substrate, or novel fluorogenic substrates) and is more sensitive than the current white blood cell (WBC) assay.

The isolated leukocytes, specifically B-lymphocytes, can be immortalized via infection with Epstein-Barr virus (EBV) to generate a replenishable lymphoblast cell lines for additional characterization. The lymphoblast cell lines provide for a new in vitro assay that is non-invasive, and also provides for a very reliable method for rapidly evaluating all known disease-causing mutations and for determining whether a SPC therapy will be effective in a patient with specific mutations.

In conjunction with genotyping, both assays provide a method for determining whether newly discovered GAA mutations (such as spontaneous mutations) cause the GAA to misfold and, are “rescuable” using SPCs.

According to the present invention. GAA enzyme activity can be measured in lysosomes in freshly isolated leukocytes or lymphoblast or fibroblast cell lines in situ to provide data on whether a patient would be responsive to SPCs. This assay can also be used to used to develop and optimize an appropriate dosing regimen for an individual patient by determining an effective dose or dosing regimens for increasing the activity of mutant GAA enzyme levels and activity in lysosomes.

The in vivo assay of the invention is a minimally-invasive method that measures GAA activity in freshly-isolated leukocytes to determine whether a patient responds to SPCs while on the test drug to qualify or dis-qualify a potential patient for SPC therapy.

The present invention further provides a method to accurately measure GAA activity in Tissue homogenate samples.

Measuring GAA activity in 1-deoxynojirimycin (DNJ) treated samples can be difficult since residual levels of this compound can inhibit GAA and lead to reduced enzyme activity measurements. The instant invention provides a new method to overcome this inhibition problem and enable accurate measurements of GAA activity in tissue homogenate samples. This method utilizes concanavalin A (Con A), a lectin protein from jack bean that binds glycoproteins via their terminal glucose and/or mannose carbohydrates. GAA, like the vast majority of other proteins that are synthesized in the endoplasmic reticulum (ER)_, contain core (also called N-linked) carbohydrates and therefore also binds this lectin. One embodiment of the invention is a method that utilizes Con A, which is covalently coupled to an insoluble matrix (e.g., agarose or sepharose) which can be sedimented by centrifugation and enable efficient washout of 1-deoxynojirimycin (DNJ) prior to GAA activity measurements. Moreover, since Con A only binds a glycoprotein via the carbohydrates, there is sufficient distance between the Con A-bound N-glycans and the enzyme active site and therefore still allows for substrate binding and catalysis. This method can be used to measure GAA activity in a number of different cell types (including wild-type and patient derived primary peripheral leukocytes, lymphoblasts, fibroblasts, myoblasts, and in transiently transfected COS-7 cells) as well as tissues homogenates (including multiple skeletal and cardiac muscles, brain, skin, etc.). Hence, this method is useful for measuring GAA activity in a broad range of cells and tissues.

Furthermore, the use of Con A can actually improve the sensitivity and accuracy of the assay by concentrating glycoproteins on a small volume. More specifically, conventional assays are performed at relatively small volumes (e.g. 100 microliters) and the amount of sample that can be added is typically only a portion of this total volume (e.g. less than half) because substrate and other reagents are added into the assay. This becomes problematic with patient-derived samples that have low residual activity because one cannot add enough sample (volume) into the assay and the signals can be only slightly above (or at or below) background which makes the data less accurate. By using Con A, essentially all of the glycoproteins can be captured, including the enzyme of interest such as the lysosomal enzymes, onto the small volume of the beads. Hence, instead of assaying only 50 microliters worth of sample due to limited volume constraints using the conventional methodology, this new method enables the capture of 1000 microliters worth of sample onto a small volume (e.g. 25 microliters) of the Con A beads (due to the beads high binding capacity) and assay these beads directly. The net result is the effective “concentration” of sample for better signals which in turn yields much more accurate enzyme activity measurements. This improved assay is particularly useful when working with patient lymphoblasts which often have 10-fold lower enzyme activity than fibroblasts and other cell types.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Pompe disease” also referred to as acid maltase deficiency, glycogen storage disease type II (GSDII), and glycogenosis type II, is a genetic lysosomal storage disorder characterized by mutations in the GAA gene which metabolizes glycogen. As used herein, this term includes infantile, juvenile and adult-onset types of the disease.

“Acid α-glucosidase or α-glucosidase or GAA” is a lysosomal enzyme which hydrolyzes alpha-1,4- and alpha-1,6-linked-D-glucose polymers present in glycogen, maltose, and isomaltose. Alternative names are as follows: glucoamylase: 1,4-α-D-glucan glucohydrolase; amyloglucosidase; gamma-amylase: and exo-1,4-α-glucosidase, and gamma-amylase. The human GAA gene has been mapped to chromosome 17q25.2-25.3 and has nucleotide and amino acid sequences depicted in GenBank Accession No. Y00839. Mutations resulting in misfolding or misprocessing of the GAA enzyme include T1064C (which changes Leu in position 355 into Pro) and C2104T (which substitutes Arg 702 into Cys) (Montalvo et at. Mol Genet Metab. 2004: 81(3): 203-8). In addition, Hermans et al. (Human Mutation 2004; 23: 47-56) describe a list of GAA mutations which affect maturation and processing of the enzyme. Such mutations include Leu405Pro and Met519Thr. In one non-limiting embodiment, the method of the present invention is expected to be useful for mutations that cause unstable folding of α-glucosidase in the ER.

The term “wild-type activity” refers to the normal physiological function of a GAA in a cell. For example. GAA activity includes folding and trafficking from the ER to the lysosome, with the concomitant ability to hydrolyze α-1,4- and α-1.6-linked-D-glucose polymers present in glycogen, maltose, and isomaltose.

The term “wild-type GAA” refers to the nucleotide sequences encoding GAA, and polypeptide sequences encoded by the aforementioned nucleotide sequences (human GAA GenBank Accession No. Y00839, and any other nucleotide sequence that encodes GAA polypeptide (having the same functional properties and binding affinities as the aforementioned polypeptide sequences), such as allelic variants in normal individuals, that have the ability to achieve a functional conformation in the ER, achieve proper localization within the cell, and exhibit wild-type activity (e.g., hydrolysis of glycogen).

A “patient” refers to a subject who has been diagnosed with a particular disease. The patient may be human or animal. A “Pompe disease patient” refers to an individual who has been diagnosed with Pompe disease and has a mutated GAA as defined further below.

As used herein the term “mutant α-glucosidase” or “mutant GAA” refers to an α-glucosidase polypeptide translated from a gene containing a genetic mutation that results in an altered α-glucosidase amino acid sequence. In one embodiment, the mutation results in an α-glucosidase protein that does not achieve a native conformation under the conditions normally present in the ER, when compared with wild-type α-glucosidase or exhibits decreased stability or activity as compared with wild-type α-glucosidase. This type of mutation is referred to herein as a “conformational mutation,” and the protein hearing such a mutation is referred as a “conformational mutant.” The failure to achieve this conformation results in the α-glucosidase protein being degraded or aggregated, rather than being transported through a normal pathway in the protein transport system to its native location in the cell or into the extracellular environment. In some embodiments, a mutation may occur in a non-coding part of the gene encoding α-glucosidase that results in less efficient expression of the protein, e.g., a mutation that affects transcription efficiency, splicing efficiency. mRNA stability, and the like. By enhancing the level of expression of wild-type as well as conformational mutant variants of α-glucosidase, administration of an α-glucosidase pharmacological chaperone can ameliorate a deficit resulting from such inefficient protein expression. Alternatively, for splicing mutants or nonsense mutants which may accumulate in the ER, the ability of the chaperone to bind to and assist the mutants in exiting the ER, without restoring lysosomal hydrolase activity, may be sufficient to ameliorate some cellular pathologies in Pompe patients, thereby improving symptoms.

Exemplary mutations of GAA include the following: D645E (Lin et al., Zhonghua Min Guo Xiao Er Ke Yi Xue Hui Za Zhi. 1996; 37(2): 115-21); D645H (Lin et al., Biochem Biophys Res Commun. 1995 17; 208(2): 886-93); R224W, S619R, and R660H (New et al. Pediatr Neurol. 2003; 29(4): 284-7); T1064C and C2104T (Montalvo et al., Mol Genet Metab. 2004:81(3): 203-8); D645N and L901Q (Kroos et al., Neuromuscul Disord. 2004; 14(6): 371-4); G219R, E262K, M408V (Fernandez-Hojas et al., Neuromuscul Disord. 2002; 12(2): 159-66); G309R (Kroos et al., Clin Genet. 1998; 53(5): 379-82); D645N, G448S, R672W, and R672Q (Huie et al., Biochem Biophys Res Commun. 1998; 27:244(3): 921-7); P545L (Hermans et al. Hum Mol. Genet. 1994; 3(12): 2213-8); C647W (Huie et al. Huie et al., Hum Mol. Genet. 1994; 3(7): 1081-7); G643R (Hermans et al. Hum Mutat. 1993; 2(4): 268-73); M318T (Zhong et al., Am J Hum Genet. 1991; 49(3): 635-45); E521K (Hermans et al., Biochem Biophys Res Commun. 1991; 179(2): 919-26); W481R (Raben et al. Hum Mutat. 1999:13(1): 83-4); and L552P and G549R (unpublished data).

Splicing mutants include IVS1AS. T>G, −13 and IVS8+1G>A).

Additional GAA mutants have been identified and are known in the art. Conformational mutants are readily identifiable by one of ordinary skill in the art.

Mutations which impair folding, and hence, trafficking of GAA, can be determined by routine assays well known in the art, such as pulse-chase metabolic labeling with and without glycosidase treatment to determine whether the protein enters the Golgi apparatus, or fluorescent immunostaining for GAA localization within the cell. Wild-type GAA is secreted as a 110 kD precursor which then converts to the mature GAA of 76 kD via and intermediate of 95 kD.

Such functionality can be tested by any means known to establish functionality of such a protein. For example, assays using fluorescent substrates such as 4-methyl umbeliferryl-α-D-glueopyranoside can be used to determine GAA activity. Such assays are well known in the art (see e.g., Hermans et al., above).

As used herein, the term “specific pharmacological chaperone” (“SPC”) or “pharmacological chaperone” refers to any molecule including a small molecule, protein, peptide, nucleic acid, carbohydrate, etc. that specifically binds to a protein and has one or more of the following effects: (i) enhances the formation of a stable molecular conformation of the protein; (ii) induces trafficking of the protein from the ER to another cellular location, preferably a native cellular location, i.e., prevents ER-associated degradation of the protein; (iii) prevents aggregation of misfolded proteins: and/or (iv) restores or enhances at least partial wild-type function and/or activity to the protein. A compound that specifically binds to e.g. GAA, means that it binds to and exerts a chaperone effect on GAA and not a generic group of related or unrelated enzymes. More specifically, this term does not refer to endogenous chaperones, such as BiP, or to non-specific agents which have demonstrated non-specific chaperone activity against various proteins, such as glycerol. DMSO or deuterated water, i.e., chemical chaperones (see Welch et al., Cell Stress and Chaperones 1996; 1(2): 109-115; Welch et al., Journal of Bioenergetics and Biomembranes 1997; 29(5): 491-502: U.S. Pat. No. 5,900,360; U.S. Pat. No. 6,270,954; and U.S. Pat. No. 6,541,195). In the present invention, the SPC may be a reversible competitive inhibitor.

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

Following is a description of some (SPC) specific pharmacological chaperones contemplated by this invention:

1-deoxynojirimycin (DNJ) refers to a compound having the following structures:

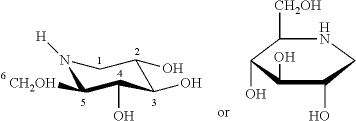

This term includes both the free base and any salt forms.

Still other SPCs for GAA are described in U.S. Pat. No. 6,599,919 to Fan et al., and U.S. Patent Application Publication US 20060264467 to Mugrage et al. and include N-methyl-DNJ, N-propyl-DNJ, N-butyl-DNJ, N-pentyl-DNJ, N-hexyl-DNJ, N-heptyl-DNJ, N-octyl-DNJ, N-nonyl-DNJ. N-methylcyclopropyl-DNJ, N-methylcyclopentyl-DNJ, N-2-hydroxyethyl-DNJ, and 5-N-carboxypentyl DNJ.

In one embodiment, the SPC is selected from N-methylcyclopropyl-DNJ and N-methylcyclopentyl-DNJ.

As used herein, the term “specifically binds” refers to the interaction of a pharmacological chaperone with a protein such as GAA, specifically, an interaction with amino acid residues of the protein that directly participate in contacting the pharmacological chaperone. A pharmacological chaperone specifically binds a target protein, e.g., GAA, to exert a chaperone effect on GAA and not a generic group of related or unrelated proteins. The amino acid residues of a protein that interact with any given pharmacological chaperone may or may not be within the protein's “active site.” Specific binding can be evaluated through routine binding assays or through structural studies, e.g. co-crystallization. NMR, and the like. The active site for GAA is the substrate binding site.

“Deficient GAA activity” refers to GAA activity in cells from a patient which is below the normal range as compared (using the same methods) to the activity in normal individuals not having or suspected of having Pompe or any other disease (especially a blood disease).

As used herein, the terms “enhance GAA activity” or “increase GAA activity” refer to increasing the amount of GAA that adopts a stable conformation in a cell contacted with a pharmacological chaperone specific for the GAA, relative to the amount in a cell (preferably of the same cell-type or the same cell. e.g. at an earlier time) not contacted with the pharmacological chaperone specific for the GAA. This term also refers to increasing the trafficking of GAA to the lysosome in a cell contacted with a pharmacological chaperone specific for the GAA, relative to the trafficking of GAA not contacted with the pharmacological chaperone specific for the protein. These terms refer to both wild-type and mutant GAA. In one embodiment, the increase in the amount of GAA in the cell is measured by measuring the hydrolysis of an artificial substrate in lysates from cells that have been treated with the SPC. An increase in hydrolysis is indicative of increased GAA activity.

The term “GAA activity” refers to the normal physiological function of a wild-type GAA in a cell. For example, GAA activity includes hydrolysis of alpha-1,4- and alpha-1.6-linked-D-glucose polymers present in glycogen, maltose, and isomaltose.

A “responder” is an individual diagnosed with Pompe disease and treated according to the presently claimed method who exhibits an improvement in, amelioration, or prevention of one or more clinical symptoms, or improvement or reversal of one or more surrogate clinical markers that are indicators of disease pathology. Symptoms or markers of Pompe disease include but are not limited to decreased GAA tissue activity: cardiomyopathy; cardiomegaly; progressive muscle weakness, especially in the trunk or lower limbs; profound hypotonia; macroglossia (and in some cases, protrusion of the tongue); difficulty swallowing, sucking, and/or feeding; respiratory insufficiency; hepatomegaly (moderate): laxity of facial muscles; areflexia; exercise intolerance; exertional dyspnea; orthopnea; sleep apnea; morning headaches; somnolence; lordosis and/or scoliosis: decreased deep tendon reflexes; lower hack pain; and failure to meet developmental motor milestones.

The dose that achieves one or more of the aforementioned responses is a “therapeutically effective dose.”

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. 18th Edition, or other editions.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

### Method

To easily determine whether SPC therapy will be a viable treatment for Pompe patients, non-invasive DNJ rescue assay of GAA activity in lymphobasts. WBCs, or subsets of WBCs, from Pompe patients was developed.

### I. Ex Vivo Assay

In one embodiment, the diagnostic method of the present invention involves isolating leukocytes (mostly B- and T-lymphocytes) from blood specimens from Pompe patients (or patients suspected of having Pompe disease). In another embodiment, the diagnostic method of the present invention involves establishing lymphoblast cell cultures from freshly-isolated B-lymphocytes for longer-term studies. Both cell model systems are then treated with or without an SPC, e.g. DNJ, lysed and assayed for the enhancement (i.e. increase) of endogenous GAA activity to determine if a patient will likely respond to SPC therapy (i.e. the patient will be a “responder”).

This embodiment can be carried out as follows.

### White Blood Cell Separation

The WBCs are prepared using standard techniques, e.g. collection, centrifugation, separation, and washing. More specifically, they can be prepared according to the following steps:
1. A blood sample is drawn from a Pompe patient. In specific embodiments, approximately 8 to 10 mL are drawn into an appropriate container such as a ACD tube from Becton-Dickenson (containing a sodium citrate anti-coagulant and a separation medium).
2. The anti-coagulated blood sample is then layered on top of dense gradient. e.g. Ficoll-Hypaque, Percoll or other similar density gradients and centrifuged to enrich B- and T-lymphocytes at the interface while pelleting red blood cells, monocytes, granulocytes, etc.
3. Half of the plasma layer is discarded (without disturbing the white blood cell layer) and remaining fluid containing white blood cells is transferred to a centrifuge tube.
4. The WBCs are then pelleted and washed for two or more times by re-suspending the pelleted cells in an appropriate isotonic buffer. e.g. PBS, followed by centrifugation for about 15-20 minutes at about 320×g.
5. The pellet is then re-suspended with a small volume of appropriate isotonic buffer. e.g. PBS. Half of the pellet is transferred to a labeled cryovial for freezing. The other half is used for establishing T cell cultures as described below. The sample that is to be frozen is centrifuged and then resuspended in a small volume of appropriate isotonic buffer. e.g. RPMI 1640 plus DMSO, prior to freezing.

### Leukocyte Cell Cultures

In one embodiment, lymphocyte cell cultures are established and expanded by stimulation with a mitogenic as follows:
1. The washed cells from above Ficoll isolation are re-suspended in an appropriate cell culture medium, such as RPMI supplemented with stimulatory cytokines and/or mitogens. Suggested stimulatory cytokines include IL-2, IL-12, IL-15 phytohemagglutinin (PHA), concanavalin A (con A), and pokeweed mitogen. In a particular embodiment, the lymphocytes are re-suspended in an appropriate volume of RPMI 1640 medium supplemented with FBS, IL-2 and a stimulatory concentration of PHA. They can then be transferred to an appropriate culture vessel and incubated for sufficient time to expand, e.g., about 2-3 days.
2. After the lymphocytes have expanded, they may be cryo-preserved (at ˜3×10⁶ cells/vial) using RPMI 1640 medium supplemented for cryopreservation medium. e.g., containing FCS and DMSO. This is sufficient to thaw 5 mL of culture at 5×10⁵ viable cells/mL.

It is noted that one of ordinary skill in the art will be able to ascertain appropriate amounts of T cell stimulatory cytokines or mitogens, although typically such agents are added at amounts from between about 1 ng/ml and about 25 ng/ml (or about 100 U/ml) for cytokines. For mitogens, concentrations range from about 10 ng/ml to about 10 μg/ml for mitogens with most being effective in the low μg/ml range.

### Lymphoblast Cell Preparation

Lymphoblastoid cell lines (LCLs) are leukocyte cultures (primarily B cells) that have been transformed with the Epstein-Barr Virus (EBV) to produce proliferative suspension cultures. Because well established LCLs can be very fast-growing (even those with genetic and metabolic disorders), their density must be carefully controlled to prevent overcrowding over an extended period. In one non-limiting embodiment, the following protocol details cell seeding density, treatment with a test compound, treatment compound washout, lysing, and assaying of LCLs for the measurement of acid α-glucoside (GAA) with the test compound.

### Enzyme Activity/Enhancement Assay

In one embodiment, T cells or lymphoblasts isolated above (e.g. approximately 2.5×10⁶) are grown in culture medium (preceded by thawing if they are frozen), in an appropriate culture vessel in the absence or presence of the SPC, e.g., DNJ, for enough time to evaluate the change in GAA activity. e.g., 2 or 3 days for T-cells and 5 days for lymphoblasts. Doses of DNJ expected to enhance GAA in T cells are in a range from about 2 nM to about 150 μM, preferably about 1 μM to 100 μM, and more preferably about 5 μM to 50 μM. In one specific embodiment, DNJ is added at about 20 μM. Doses of DNJ expected to enhance GAA in lymphoblasts are in a range from about 2 nM to about 300 μM, preferably about 1 μM to 100 μM, and more preferably about 5 μM to 50 μM. In one specific embodiment. DNJ is added at about 30 μM. Cells can be harvested by centrifugation and washed twice with PBS. Pellets can be stored frozen at −80° C. until assayed for enzyme activity.

Cells are then lysed by the addition of lysis buffer, which contains 150 mM NaCl, 25 mM Bis-Tris and 0.1% Triton-X100 (or deionized water) and physical disruption (pipetting, vortexing and/or agitation, and/or sonication) at room temperature or on ice, followed by pooling of the lysates on ice, then splitting the pooled lysate into small aliquots and freezing.

The lysates can be thawed immediately prior to the assay and should be suspended by use of a vortex mixer and sonicated prior to addition to appropriate wells e.g., in a microplate. 4-methyl umbeliferryl-α-D-glucopyranoside (4MU-alphaGlc), or other appropriate labeled DNJ substrate, is then added and the plate is gently mixed for a brief period of time, covered, and incubated at 37° C. for a sufficient time for substrate hydrolysis, usually about 1 hour. To stop the reaction. NaOH-glycine buffer (alternatively sodium carbonate), pH 10.7, is added to each well and the plate is read on a fluorescent plate reader (e.g. Wallac 1420 Victor™ or similar instrument). Excitation and emission wavelengths were customarily set at 355 nm and 460 nm, respectively. One unit of enzyme activity is defined as the amount of enzyme that catalyzes the hydrolysis of 1 nmole of 4-methylumbelliferone per hour. For each patient sample at least three normal samples should be tested concurrently.

Various modifications of this assay will be readily ascertainable to one of ordinary skill in the art. Examples of artificial substrates that can be used to detect GAA activity include but are not limited to 4MU-alphaGlc. Obviously, only substrates that can be cleaved by human GAA are suitable for use. It is noted that while use of a fluorogenic substrate is preferred, other methods of determining GAA activity are contemplated for use in the method, including using chromogenic substrates or immunoquantification techniques.

In an alternative embodiment, the ability of a SPC to enhance the activity of GAA in a lymphoblast cell line (LCL) can be determined as described in the following, non-limiting example:

Seeding
All cell culture work can be performed in a BLII Bio-safety cabinet using sterile techniques. LCL culture is expanded to a T75 by transferring 7×10⁶-1×10⁷ cells total to a T75 and add 40 ml 37° C. complete growth media.
Optimal LCL cultures are selected in a T75 flasks based on cell density and viability. Cell count can be performed on these cultures. In one embodiment, a cell density of 1×10⁶ cells/ml maintains LCLs at the highest viability (e.g. 90-98% viable). Cell densities higher than 1×10⁶ cells/ml can drastically reduce the overall viability in the culture.
A sterile 50 ml conical centrifuge tube can be used to prepare a cell suspension in the appropriate amount of complete growth media to obtain a final cell density of, for example, approximately 2.0×10⁵ cells/ml at a volume of at least 20 ml. In one non-limiting example, if the original culture contains 1×10⁶ cells/ml, 16 ml media should be added to 4 ml of the cell suspension in the staging tube to create a density of 2×10⁵ cells/ml in a volume of 20 ml. Dispense into four labeled T25 flasks at 5 ml each total volume. This process is repeated for each LCL to be processed. Place all flasks in a humidified 5% CO₂ 37° C. incubator overnight. The original T75 cultures can be expanded and returned to the same incubator, if necessary.

Treatment with Test Compound
Treatment of the cells with a test compound, for example, DNJ, is performed 24 hours after they are seeded into T25 flasks.
in one embodiment. 5 ml of each treatment concentration is required for each cell line. Cell lines are treated with the test compound over a concentration range of 0, x, 3x, and 10x test compound.
A 2x stock test compound solution can be prepared in, for example, sterile 15 ml or 50 ml centrifuge tubes for each condition and add 5 ml solution to 5 ml pre-incubated culture. In one non-limiting example, when using DNJ for the treatment of GAA, stock concentrations of 0, 60, 200, and 600 μM DNJ made; when added to the flasks, the final concentrations are 0, 30, 100, and 300 μM DNJ.
Each set of flasks are then marked with the appropriate treatment concentrations, and add, for example, 5 ml from the corresponding stock suspension to each flask. Return all flasks to the incubator for 5 days.

Overnight Compound Washout
After five days (120 hours) of treatment, exchange 100% of the media in cell suspension with compound-free complete media in the following manner;
Transfer the contents of each T25 to a sterile 15 ml conical centrifuge tube that has been pre-numbered to maintain order of concentration range.
When each set is transferred, wash each T25 with 5 ml blank RPMI 1640 (no phenol red).
Centrifuge the tubes at 21° C. for 10 min at 600 g. During the spin, remove the blank RPMI from the flasks by aspiration, maintaining sterility.
Following the centrifugation, remove the supernatant from the tubes by aspiration and resuspend the pellets in 10 ml complete media. Transfer the cell suspensions back to their respective flasks. Return all flasks to the 5% CO₂ 37° C. incubator overnight.

Cell Lysis
16-24 hours after the compound washout, prepare a cell lysis solution by adding 5 complete-mini protease inhibitor tablets to 50 ml GAA lysis buffer and let dissolve at room temperature by gentle inversion.
Collect the LCLs from each flask and transfer to a sterile 15 ml conical centrifuge tube.
Spin the tubes at 21° C. at 600 g. for 10 min.
After centrifugation, remove the supernatant and resuspend the cell pellet by pipette with 5 ml 1×PBS at room temperature. Spin at 600 g for 10 min at room temperature.
Following the PBS wash, remove the supernatant by aspiration and add 1.5 ml GAA lysis buffer with protease inhibitors (previously prepared).
Use a p1000 micropipette set at 1 ml to gently and completely resuspend the pellet in lysis buffer without creating bubbles or foam.
Spin the tubes at room temperature for 5 min at 800 g and store room temperature.

Assay
Transfer the lysate supernatants to 96-well cluster tube racks. Each lysate can be added to one tube of the cluster rack.
The lysates can be stored without any affect on the activity for up to two weeks at 4° C. in the cluster tube racks with caps.

Protein Assay (micro-BCA)
The protein determination in the cell lysis supernatants is performed using the Pierce micro-BCA kit (Pierce#23235). Use black 96-well flat-bottom plates for the BCA assay.
In a 96-well plate create a BSA serial dilution in the following manner. Add 100 μl diH₂O to rows A and B (24 wells total). Add 100 μl of the 2 mg/ml BSA solution provided in the kit to wells A1 and B1 and mix by pipetting. Transfer 100 μl from A1 to A2 and mix by pipetting, then transfer 100 μl from A2 to A3. Continue in this manner for the rest of row A, repeating the process for row B.
In a separate black plate, add 130 μl diH₂O to all standard, blank, and sample wells to be used.
Transfer 20 μl of the BSA serial dilution to rows A and B.
Add 20 μl GAA lysis buffer to row C as a blank.
Add 20 μl from each sample into duplicate wells as shown in the plate map.
Add 150 μl BCA reaction reagent (included in the micro-BCA kit: 25 ml
Reagent A, 24 ml Reagent B, and 1 ml Reagent C) to all standard, blank, and sample wells.
Incubate the plate at 37° C. for two hours.
Following incubation, measure the absorbance of the plates on the multi-well plate reader at A550 nm. Convert these data using pre-made templates in Excel to calculate the concentration of protein in each lysate. This will be used along with the 4-MU activity calculation to determine 4-MU released per μg protein per hour.

GAA Activity Assay
Each assay day prepare fresh (within one hour of use) a solution of 1 mg/ml 4-Methylumbelliferyl-alpha-D-glucopyranoside by adding 250 μl DMSO to 25 mg of the substrate at room temperature and dissolving it by vortexing. Then add the solution to 25 ml GAA reaction buffer (67 mM potassium acetate (with glacial acetic acid) pH 4.0) in a 50 ml conical centrifuge tube and keep in the dark.
In a black 96-well tissue culture plate add 75 μl of the substrate solution prepared above at room temperature to all sample and blank wells.
Add 25 μl GAA lysis buffer to row G to serve as the blank.
Finally, add 25 μl of each lysate to rows A-F in each column. Each lysate will be placed in six separate wells one lysate per column. Up to three cell lines can be assayed in the same plate. Incubate the plates at 37° C. for two hours.
After the incubation, remove the plates from the incubator and stop the reaction by adding 100 μl 0.5 M sodium carbonate to all samples and the blank wells.
A 4-MU standard curve will be generated in row H of each plate: add 50 μl 0.5M sodium carbonate and 50 μl GAA reaction buffer to row H. Then add 100 μl of a 15 μM solution of 4-methylumbelliferone to wells H1 and H7. Serially dilute these wells at a ratio of 1:2 for a total of 6 points each (H1 through H6; and H7 through H12 as a duplicate).
Read the plates on a multi-well plate reader using 355 nm emission and 460 nm excitation filters. Convert the data using pre-made templates in Excel to calculate nmol 4-MU released per mg total protein per hour using the protein concentration determined via the BCA protein assay.

Diagnosis and Prognosis. The T cell or lymphoblast assay can be easily modified for use as a diagnostic assay to diagnose Pompe disease by simply eliminating the step of culturing the T cells or lymphoblasts in the presence of DM prior to performing the enhancement assay. The activity of GAA in T cells or lymphoblast established from an individual suspected of having Pompe disease can instead be quantitated using T cells or lymphoblast from a normal individual as a control. Moreover, both GAA activity and SPC enhancement assays can be performed almost simultaneously using the same T cells or lymphoblasts derived from one patient sample. While not being bound thereby, it is believed that since T cells may express more GAA (GAA in normal T cells as compared with WBCs is much higher), it will be easier to confirm with more certainty whether a patient has GAA activity below the normal range because the margin of error will be smaller. Accordingly, use of the cell assay could potentially prevent misdiagnoses.

In addition, the modified assay also can be used to periodically monitor the progress of patients in whom SPC therapy was initiated to confirm that GAA activity remains increased relative to prior to treatment initiation.

### II. In Vivo Assay

In a second embodiment. WBCs are evaluated for GAA enhancement by an SPC in vivo. In this embodiment, GAA activity in WBCs derived from patients is assessed prior to SPC administration, in order to obtain a baseline value. Patients are then administered DNJ daily 2500 mg/day) for a sufficient time period, e.g., about 10 days to about 2 weeks, followed by extraction of blood and determination of changes in GAA activity from the baseline value. Culturing the cells either prior to, or following administration, is not required.

The dose and dosing regimen of DNJ administration during the in vivo evaluation period may vary depending on the patient since there is so much heterogeneity among mutations, and depending on the patient's residual GAA activity. As a non-limiting example, the doses and regimens expected to be sufficient to increase GAA in most “rescuable” individuals is as described in U.S. Provisional Application 61/028,105, filed Feb. 12, 2008, herein incorporated by reference in its entirety.

Administration of DNJ according to the present invention may be in a formulation suitable for any route of administration, but is preferably administered per os in an oral dosage form such as a tablet, capsule or solution. For this assay, in the case of oral administration, it is preferred that the patient be administered the DNJ without food (e.g., no food 2 hours before and for 2 hours after dosing) since bioavailability may be lower if taken with food, thereby risking inaccurate results.

Patients who are on other therapies, such as ERT, may wish to cease treatment for at least about 28 days prior to the in vivo assay to ensure the most accurate results.

### White Blood Cell Separation

WBCs are isolated and separated as described above for the T cell in vitro assay. However, no RPMI media or DMSO is to be added to the pellets prior to freezing (as per step 5 in the section entitled “White Blood Cell Separation” above).

### Enzyme Activity/Enhancement Assay

Pellets are thawed on ice and cells are then lysed by the addition of lysis buffer and physical disruption (such as by use of a vortex mixer and agitation, and/or sonication at room temperature) for a sufficient time, followed by pooling of the lysates in a polypropylene tube on ice, then splitting of the pooled lysate into aliquots for freezing.

The WBC lysates are then thawed on ice and mixed (again, by sonication and/or vortexing). Samples of each lysate, as well as standards and negative controls, are then added to appropriate wells in e.g. a 24 or 96 well microplate. A labeled substrate, such as, for example, 4MU-alphaGlc in citrate/phosphate buffer, pH 4.6, is then added to all wells, and incubation for a short time at ambient temperature. The plate is then mixed briefly and incubated at 37° C. for a sufficient time period to permit substrate hydrolysis, e.g., about 1 hour. After the sufficient time period, the reaction is stopped by the addition of stop buffer and the plate is read on a fluorescent plate reader (e.g. Wallac 1420 Victor3™) to determine enzyme activity per well.

Various modifications of this assay will be readily ascertainable to one of ordinary skill in the art. Examples of artificial substrates that can be used to detect GAA activity include but are not limited to 4MU-alphaGlc. Obviously, only substrates that can be cleaved by human GAA are suitable for use. It is noted that while use of a fluorogenic substrate is preferred, other methods of determining GAA activity are contemplated for use in the method, including using chromogenic substrates or immunoquantification techniques.

### Eligibility Determination Criteria

The criteria for determining eligibility for SPC therapy depends on the patient's residual enzyme activity at baseline. i.e., the activity determined in the untreated T cells or lymphoblast in the in vitro assay, or the activity in the WBCs prior to SPC administration in the in vivo assay. The lower the residual activity, the greater enhancement necessary in order for a patient to be considered a likely responder to treatment.

In one embodiment, the criteria for determining eligibility for the in vitro assay are as follows:
If baseline GAA activity in lymphocytes or lymphoblasts is less than a specified value (e.g. 1% of normal), then GAA activity after incubation with DNJ must be at least twice that of the specified value (e.g. 2% of normal);
If baseline GAA activity in lymphocytes or lymphoblasts is between specified values (e.g. between 1% of normal and <3% of normal), then GAA activity after incubation with DNJ must be at least two times a specified value (e.g., the baseline level);
If baseline GAA activity in lymphocytes or lymphoblasts is between specified values (e.g. between 3% of normal and <10% of normal), then GAA activity after incubation with DNJ must be at least 3% of a normal higher than the baseline level; and
If baseline GAA activity in lymphocytes or lymphoblasts is more than a specified value (e.g. 10% of normal or more), then GAA activity after incubation with DNJ must be at least 1.3 times a specified value (e.g. 1.3 times the baseline level).

In one embodiment, for the in vivo assay, the following criteria are used to determine eligibility criteria:
If baseline GAA is less than a specified value (e.g. 1% of normal), then Day 15 GAA activity after treatment with DNJ must be at least twice that of the specified value (e.g. 2% of normal):
If baseline GAA is between specified values (e.g. between 1% of normal and <5% of normal), then GAA activity must be at least two times a specified value (e.g. a baseline level) following the treatment period;
If baseline GAA is between specified values (e.g. between 5% of normal and <10% of normal), then GAA activity must be at least 5% of normal higher than the baseline level following the treatment period; and
If baseline GAA more than a specified value (e.g. 10% of normal or more, then GAA activity must be at least 1.5 times a specified value (e.g. 1.5 times the baseline level) following the treatment period.

In an alternative embodiment, an increase in activity of at least about 20% in the cells cultured with SPC over the activity in the cells not cultured with SPC, in either the in vitro or in vivo assay, may be indicative that the patient will have a clinically relevant (therapeutically effective) response to SPC therapy.

This discovery provides a method for improving the diagnosis of and facilitating clinical treatment decisions for Pompe disease in particular, and lysosomal storage disease in general. Moreover, this method can be extended to a wide range of genetically defined diseases in appropriate cell types. This class of disease includes the other lysosomal storage disorders. Cystic Fibrosis (CFTR) (respiratory or sweat gland epithelial cells), familial hypercholesterolemia (LDL receptor; LPL-adipocytes or vascular endothelial cells), cancer (p53: PTEN-tumor cells), and amyloidoses (transthyretin) among others.

### Kits

The present invention also provides for a commercial diagnostic test kit in order to make therapeutic treatment decisions. The kit provides all materials discussed above and in the Example below, for preparing and running each assay in one convenient package, with the obvious exception of patient blood, optionally including instructions and an analytic guide.

As one non-limiting example, a kit for evaluating GAA activity may contain, at a minimum:
a. at least one T cell stimulatory agent;
b. a specific pharmacological chaperone; and
c. a chromogenic or fluorogenic substrate for the enzyme assay (including an appropriate standard)
   The kit may also contain instructions for optimally performing the protein enhancement assay. In another embodiment, the kit will contain the appropriate tubes, buffers (e.g. lysis buffer), and microplates.

In one embodiment, the SPC is supplied in dry form, and will be re-constituted prior to addition.

In another embodiment, the invention provides a it for the diagnosis of Pompe disease. In this embodiment, the SPC is not included in the kit and the instructions are tailored specifically to diagnosis.

Patients that test positive for enzyme enhancement with an SPC can then be treated with that agent, whereas patients who do not display enzyme enhancement with a specific agent can avoid treatment which will save money and prevent the emotional toll of not responding to a treatment modality.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

### In Vitro/Ex Vivo Method for Evaluating Effects of an SPC on GAA Activity

The present Example provides an in vitro diagnostic assay to determine a Pompe patient's responsiveness to a specific pharmacological chaperone, wherein the response of patient derived lymphoblasts to DNJ was determined ex vivo. This assay may also be performed using patient derived fibroblasts.

### A. Patient Population

The ex vivo study included 14 males and 12 females with late-onset GSD-II. 3 male juveniles with GSD-II (5, 11, and 12 yrs), and 1 female infant (1 yr) with GSD-II. Patients ranged in age from 1 to 72 years; 19 of 30 patients were receiving enzyme replacement therapy (ERT status for 3 patients is unkown) and blood was drawn immediately prior to enzyme infusion. All adult and juvenile patients had at least 1 copy of the common splicing mutation (IVS1 13T>G) or a missense mutation. 23/23 adults and 2/3 juveniles had one copy of the IVS1 13T>G mutation. 8/23 adults and 2/3 juveniles had at least 1 copy of a missense mutation.

B. Preparation of Patient Derived Lymphoblast Cells, and Treatment with DNJ

Lymphoblast cell lines were derived from 26 patients and treated with DNJ (0, 30, 100 and 300 μM) for five days. Lymphoblastoid cell lines (LCLs) are leukocyte cultures (primarily B cells) that have been transformed with the Epstein-Barr Virus (EBV) to produce proliferative suspension cultures. Leukocyte cultures were prepared as described in Example 2, and transformed with the EBV to establish the lymphoblast cells. Because well established LCLs can be very fast-growing (even those with genetic and metabolic disorders), their density must be carefully controlled to prevent overcrowding over an extended period. The following protocol details cell seeding density, treatment with a test compound (i.e. DNJ) treatment compound washout, lysing, and assaying of LCLs for the measurement of acid α-glucoside (GAA) with the test compound.

### 1. Supplies

T25 flasks—4 for each cell line to be processed (BD#353136.353109)
Sterile pipettes
Micropipette (single and multi-channel) and sterile tips
Sterile 15 ml and 50 ml conical centrifuge tubes (BD#352098, 352097)
Sterile aspiration pipettes
Micro-BCA kit (Pierce#23235)
96-well cluster tube rack (Costar#4413)
96-well black flat-bottom culture plates (Costar#3603)
96-well clear flat-bottom culture plate (Costar#353072)

### 2. Reagents

RPMI 1640 (with L-glutamine; Mediatech, Herndon, VA#410040CV)
RPMI 1640 (without phenol red, Mediatech#17105CV)
Fetal Bovine Serum (FBS, heat-inactivated, sterile filtered: Mediatech#35011CV)
1×PBS (Mediatech#21040CV)
Test Compound (Amicus Chemistry Dept.)
4-methylumbelliferyl-α-D-glucopyranoside (4-MUG-α, Melford#M096)
4-methylumbelliferone (4-MU, Sigma#M1381)
Dimethyl sulfoxide (DMSO, Sigma#D2650)
0.5 M sodium carbonate (Sigma#57795)
Complete-mini protease inhibitors (Roche#11836153001)
GAA Lysis buffer comprises:
   150 mM NaCl (Fisher#S271)
   25 mM Bis-Tris (Sigma#B9754)
   0.1% Triton-X100 (Sigma#T9284)
GAA reaction buffer
   67 mM potassium acetate (with glacial acetic acid) pH 4.0
      Fisher#P250 (KOH)
      Fishery A38 (HOAc-glacial)
Complete Growth Media
   RPMI 1640 with 10% FBS and 1% L-glutamine

### 3. Equipment

5% CO₂ 37° C. humidified incubator (Thermo 3110 Series II)
Refrigerated centrifuge (Fisher#13-100-581 Accuspin 1R)
Wallac Victor³ plate reader (Perkin-Elmer#1420-012)
Biohazard level II Biosafety cabinet

### 4. Seeding

1. All cell culture work was performed in a BLII Bio-safety cabinet using sterile techniques. LCL culture was expanded to a T75 by transferring 7e⁶-1e⁷ cells total to a T75 and add 40 ml 37° C. complete growth media.
2. Optimal LCL cultures were selected in T75 flasks based on cell density and viability. A cell count was performed on these cultures. Usually a cell density of 1e⁶ cells/ml maintains LCLs at the highest viability (90-98% viable). Cells densities higher than 1e⁶ cells/ml drastically reduce the overall viability in the culture.
3. A sterile 50 ml conical centrifuge tube was used to prepare a cell suspension in the appropriate amount of complete growth media to obtain a final cell density of approximately 2.0×10⁵ cells/ml at a volume of at least 20 ml. (For example, if the original culture contains 1e⁶ cells/ml, 16 ml media should be added to 4 ml of the cell suspension in the staging tube to create a density of 2e⁵ cells/ml in a volume of 20 ml.). Cell suspensions were dispensed into four labeled T25 flasks at 5 ml each total volume. This process is repeated for each LCL to be processed. All flasks were placed in a humidified 5% CO₂ 37° C. incubator overnight. The original T75 cultures can be expanded and returned to the same incubator, if necessary.
   5. Treatment with Test Compound
1. Treatment of the cells with the DNJ test compound was performed 24 hours after the cells were seeded into T25 flasks.
2. 5 ml of each treatment concentration is required for each cell line. Cell lines were treated with test compound over a concentration range of 0, x, 3x, and 10x test compound.
3. A 2x stock of test compound solution was prepared in sterile 15 ml or 50 ml centrifuge tubes for each condition and 5 ml solution was added to 5 ml pre-incubated culture. Stock concentrations of 0, 60, 200, and 600 μM DNJ were made; when added to the flasks, the final concentrations were 0, 30, 100, and 300 μM DNJ.
4. Each set of flasks were marked with the appropriate treatment concentrations, and 5 ml from the corresponding stock suspension were added to each flask. All flasks were returned to the incubator for 5 days.

### 6. Overnight Compound Washout

1. After five days (120 hours) of treatment, 100% of the media was exchanged in the cell suspension with compound-free complete media in the following manner;
2. The contents of each T25 was transferred to a sterile 15 ml conical centrifuge tube that had been pre-numbered to maintain order of concentration range.
3. After each set was transferred, each T25 was washed with 5 ml blank RPMI 1640 (no phenol red).
4. The tubes were centrifuged at 21° C. for 10 min at 600 g. During the spin, the blank RPMI was removed from the flasks by aspiration, maintaining sterility.
5. Following the centrifugation, the supernatant was removed from the tubes by aspiration and the pellets were resuspended in 10 ml complete media. The cell suspensions were transferred back to their respective flasksAll flasks were returned to the 5% CO₂ 37° C. incubator overnight.

### 7. Cell Lysis

1. 16-24 hours after the compound washout, a cell lysis solution was prepared by adding 5 complete-mini protease inhibitor tablets to 50 ml GAA lysis buffer and dissolved at room temperature by gentle inversion.
2. The LCLs from each flask were collected and transfer to a sterile 15 ml conical centrifuge tube.
3. The tubes were spun at 21° C. at 600 g for 10 min.
4. After centrifugation, the supernatant was removed and the cell pellet was resuspended by pipetting with 5 ml 1×PBS at room temperature. The suspension was spun at 600 g for 10 min at room temperature.
5. Following the PBS wash, supernatant was removed by aspiration and 1.5 ml GAA lysis buffer with protease inhibitors was added (previously prepared).
6. A p1000 micropipette set at 1 ml was used to gently and completely resuspend the pellet in lysis buffer without creating bubbles or foam.
7. The tubes were spun at room temperature for 5 min at 800 g and store room temperature.

### 8. Assay

1. The lysate supernatants were transferred to 96-well cluster tube racks. Each lysate was added to one tube of the cluster rack.
2. The lysates can be stored without any affect on the activity for up to two weeks at 4° C. in the cluster tube racks with caps.

9. Protein (micro-BCA)
1. Protein determination in the cell lysis supernatants was performed using the Pierce micro-BCA kit (Pierce#23235). Black 96-well flat-bottom plates were used for the BCA assay.
2. In a 96-well plate a BSA serial dilution was created in the following manner, 100 μl diH₂O was added to rows A and B (24 wells total). 100 μl of the 2 mg/ml BSA solution provided in the kit was added to wells A1 and B1 and mixed by pipetting. 100 μl from A1 was transferred to A2 and mixed by pipetting, then 100 μl from A2 was transferred to A3. This was continued for the rest of row A, and the process repeated for row B.
3. In a separate black plate, 130 μl diH₂O was added to all standard, blank, and sample wells to be used.
4. 20 μl of the BSA serial dilution was transferred to rows A and B.
5. 20 μl GAA lysis buffer was added to row C as a blank.
6. 20 μl from each sample was added into duplicate wells as shown in the plate map.
7. 150 μl BCA reaction reagent (included in the micro-BCA kit: 25 ml Reagent A, 24 ml Reagent B, and 1 ml Reagent C) was added to all standard, blank, and sample wells.
8. The plate was incubated at 37° C. for two hours.
9. Following incubation, the absorbance of the plates was measured on the multi-well plate reader at A550 nm. The data was converted using pre-made templates in Excel to calculate the concentration of protein in each lysate. This was used along with the 4-MU activity calculation to determine 4-MU released per μg protein per hour.

10. GAA Activity
1. Within one hour of use, a solution of 1 mg/ml 4-Methylumbelliferyl-alpha-D-glucopyranoside was prepared by adding 250 μl DMSO to 25 mg of the substrate at room temperature and dissolved by vortexing. The solution was added to 25 ml GAA reaction buffer in a 50 ml conical centrifuge tube and kept in the dark.
2. In a black 96-well tissue culture plate 75 μl of the substrate solution prepared above was added at room temperature to all sample and blank wells.
3. 25 al GAA lysis buffer was added to row G to serve as the blank.
4. Finally, 25 μl of each lysate was added to rows A-F in each column. Each lysate was placed in six separate wells—one lysate per column. Up to three cell lines can be assayed in the same plate. The plates was incubated at 37° C. for two hours.
5. After the incubation, the plates were removed from the incubator and the reaction stopped by adding 100 μl0.5 M sodium carbonate to all samples and the blank wells.
6. A 4-MU standard curve was generated in row H of each plate: 50 μl 0.5 M sodium carbonate and 50 μl GAA reaction buffer was added to row H. Then 100 μl of a 15 μM solution of 4-methylumbelliferone was added to wells H1 and H7. These wells were serially diluted at a ratio of 1:2 for a total of 6 points each (H1 through H6: and H7 through H12 as a duplicate).

7. The plates were read on a multi-well plate reader using 355 nm emission and 460 nm excitation filters. The data was converted using pre-made templates in Excel to calculate nmol 4-MU released per mg total protein per hour using the protein concentration determined via the BCA protein assay.

### 11. Data Analysis

1. A combination of at least 3 experiments (n=3) was required for all cell lines.
2. Statistical analysis was performed using a one way ANOVA with a Dunnet's Multiple Comparison test with a 95% confidence interval measuring the significance of any enhancement of the samples with test compound versus the untreated sample.

### Results

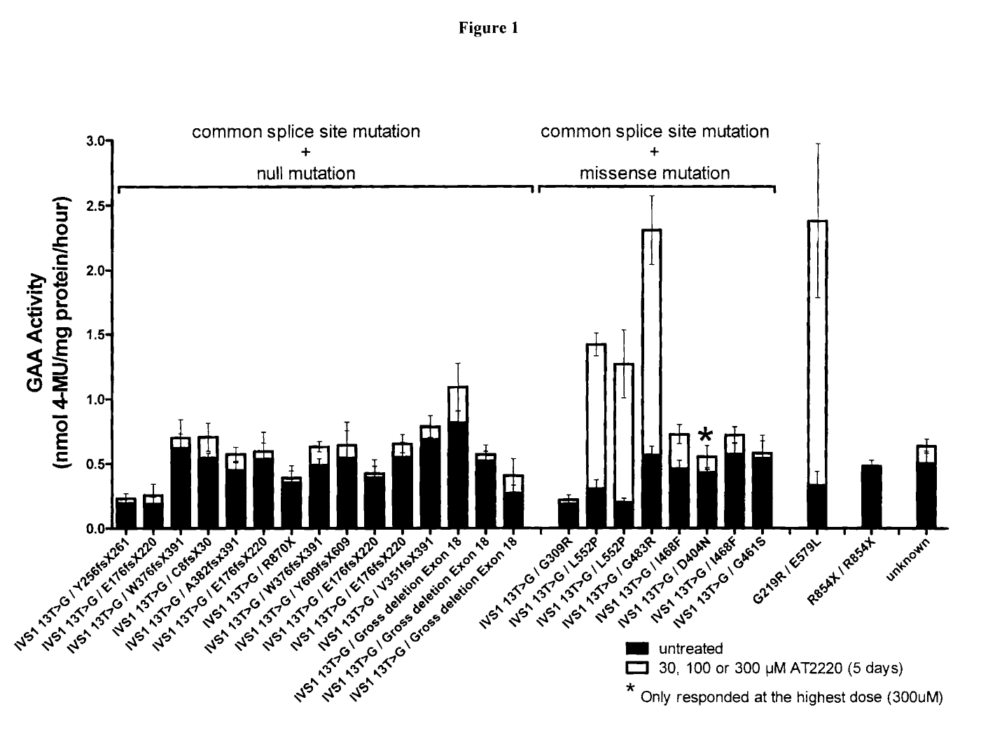

Patient-derived lymphoblasts demonstrated a dose-dependent increase in GAA levels for 24/26 patient cell lines (mean=93%: range=7-620%) and 4/24 reached significance as determined by 1 way ANOVA and Dunnett's Multiple Comparison Test (p value <0.05) (FIG. 1). DNJ increased GAA levels by 7-51% (mean=22%) in patient cell lines with one copy of the IVS1 13T>G mutation and one copy of a non-missense null mutation for GAA. Patient-derived cell fines with at least one missense mutation demonstrated a dose-dependent increase in GAA levels of 7-620% (mean=219%). While the effect of DNJ on the IVS1 13T>G mutation was small in this short 5 day treatment study, treatment of wild type mice and cynomolgus monkeys for longer periods of time (4-13 weeks) results in a ≧2-fold increase in GAA levels.

### Discussion

The present invention provides a method for establishing Lymphoblast cultures from fresh blood of normal control individuals and patients with Pompe disease. These cultures can be grown for use in an enhancement assay for GAA. These data also show that the effectiveness of GAA enhancement was evident after about 5 days in the lymphoblast growth media. The data generated are a reproducible measure of the degree of enhanced enzyme activity by a SPC for a specific genotype.

As mentioned above, this assay can also be performed using patient derived fibroblasts. In a specific embodiment, the assay using patient derived fibroblasts will be seeded in 6-well plates and be harvested using trypsin.

This method can be used for other SPC-based enhancement assays of other genetic diseases including glycosphingolipidoses and mucopolysaccharidoses, and can be extended as a research and clinical protocol in a wide range of genetically defined diseases, such as Cystic Fibrosis (CFTR) and cancer (p53, PTEN), among others.

### Prophetic Example 2

### In Vitro Method for Evaluating Effects of an SPC on GAA Activity

The present Example provides an in vitro diagnostic assay to determine a Pompe patient's responsiveness to a specific pharmacological chaperone.

### A. Preparation of Human WBC Pellets for Growth of T Lymphocytes

1. Materials:
CPT tube: Becton-Dickenson (BD Vacutainer® CPT™ Cell Preparation Tube with Sodium Citrate, cat#362761).
Human IL-2 (recombinant). PreProTECH, cat#200-02
Phytohemagglutinin (M Form) (PHA), liquid, Invitrogen, cat#10576-015
RPMI-1640 medium. Mediatech Inc., cat #10-040-CV
Fetal Bovine Serum, Mediatech Inc., cat#35-010-CV
Citric acid, monohydrate, ACS, Mallinckrodt, cat#0627
Sodium phosphate dibasic (Na₂HPO₄). ACS, Mallinckrodt cat#7917
Sodium hydroxide, volumetric solution 10N. Mallinckrodt cat#H385
Phosphoric acid, ACS, Mallinckrodt cat g PX0995-3
4-methyl umbeliferryl-α-D-glueopyranoside (4MU-alphaGle), Melford#M1096
4-methylumbelliferone (4-MU). Sigma cat#M-1381
Glycine, tissue culture grade, Fisher cat#BP381
Double deionized water
Dulbecco's Phosphate Buffered Saline. PBS, (without Ca, without Mg), Mediatech Inc. cat#21-031-CV
Micro BCA Protein Assay Kit, Pierce cat#23235
96-well microtiter plates, Costar black polystyrene 96 well round bottom, cat#3792
Costar 24-well tissue culture treated microplates. Corning Life Sciences, cat#3526
15 mL polypropylene Falcon tube, Becton Dickinson, cat#352097
Sterile Cryovials
Humidified 5% CO₂, 37° C. incubator
37° C. water bath
Fluorescence plate reader

2. WBC Separation:
Patient blood will be drawn into an 8 mL CPT tube, which has been stored at 18-25° C.
Immediately after collecting blood, it will be mixed by inverting the tube 8-10 times.
The tube will be centrifuged at room temperature (18-25° C.) for 30 minutes at 1800×g using a tabletop centrifuge equipped with swinging buckets. Universal precautions for handling blood specimens will be taken, including the use of a closed canister type bucket for centrifugation.
Following centrifugation, several layers of the blood composition will become distinguishable which represented separation of the red blood cells from the plasma and white cells. If this does not occur, warm in hands for 5 minutes and centrifuge again.

3. Washing of WBC's
Half of the plasma layer will be aspirated by vacuum and discarded without disturbing the white cell layer. All of the remaining fluid, including the cell layer, will be transferred with a Pasteur pipette to a 15 mL conical screw-cap Falcon centrifuge tube.
PBS will be added to bring the volume up to 14 mL and the tube will be mixed by inversion.
The tube will be centrifuged at room temperature for 20-30 minutes at 1300 rpm (approximately 320×g).
Immediately after centrifugation, as much supernatant as possible will be aspirated by vacuum and discarded without disturbing the cell pellet.

4. Optional Wash
The cell pellet will be re-suspended in the remaining liquid by tapping against the bottom of the tube.
10 mL of PBS will be added to the re-suspended cells, and centrifuged at room temperature for 20 minutes at 1300 rpm.
Immediately after centrifugation, as much supernatant as possible will be aspirated by vacuum and discarded without disturbing the cell pellet.

5. Optional: Freezing, WBC Pellet
The cell pellet will be mixed in the remaining liquid by tapping a finger against the bottom of the tube.
0.5 to 1 mL of PBS will be added to the re-suspended cells and one half of the pellet will be transferred (using a sterile tip on a micropipette) to a labeled 1.8 mL cryovial.
The cryovial will be centrifuged at room temperature for 5 minutes at 5000 rpm (approximately 2250 g) in a microcentrifuge.
All of the supernatant liquid will be discarded using a Pasteur pipette without disturbing the cell pellet.
0.5 to 1 ml of RPMI 1640 containing 10% FBS and 5% DMSO will then be added to the tube and mixed a pipette and frozen overnight at −80 C. prior to transferring to a liquid nitrogen cell storage freezer.
   B. Establishment of T-cell Cultures from Blood Specimens
1. The washed cells will be re-suspended in 3.0 ml of RPMI 1640 medium with 10% Cosmic Calf Serum (CCS, Hyclone Laboratories, Logan, Utah), about 25 ng/ml IL-2 (PreProTECH, Rocky Hill, N.J.) and the manufacturer's recommended concentration of PHA (Life Technology, Gaithersburg, MD). The cells will then be transferred to an upright an upright 25 cm³ culture flask and incubated for 3-4 days at 37° C., 5% CO₂.
2. The cell culture will be diluted to 5 ml with growth medium (RPMI-1640, 10% FBS, 25 ng/ml IL-2). The cell concentration will then be adjusted to about 5×10⁵ cells/ml in the flask.
3. The growth of the cells will be monitored daily, Cells will be maintained between 5×10⁵) and 1.5×10⁶ cells in an upright flask. The depth of the medium in the flask will not exceed 1 cm (about 7 mLs in a T25 and 20 mLs in a T75). Cultures can be maintained for approximately 21 days with a doubling time of about 24 hrs. Senescence of the culture will be apparent by a dramatic reduction in growth rate. Culture time may possibly be extended by re-stimulation with PHA.
4. Optional-Freezing T-lymphocytes: T-lymphocytes may be frozen at 3×10⁶ cells/vial using RPMI1640 medium containing 20% FCS and 7.5% DMSO. On day 5, 6, or 7 cryopreserve as many vials as possible at 3×10⁶ cells/vial. This is sufficient to thaw 5 mLs of culture at 5×10⁵) viable cells/ml.

When establishing T-cell cultures, the following should be noted.
Fresh blood specimens should be collected in heparinized tubes (or tubes containing an appropriate anti-coagulant) and used the same day. ACD tubes should be used if specimens cannot be processed within 24 hours. (Clin Chem 1988 January; 34(1):110-3: Clin Diagn Lab Immunol. 1998 November; 5(6):804-7.).
Eight-10 mLs of blood is usually sufficient to establish 20 million cells by day 5.
T lymphocytes are the specific targets of the HIV virus. Use extreme care if the HIV status of the patient is unknown.
Each new lot of IL-2 should be tested to determine the optimal concentration. The lot from PreProTECH used for these experiments was been found to be optimal at 25 ng/ml with only a slight reduction in cell growth at concentrations up to 50 ng/ml.
Each lot of mitogen, e.g., phytohemagglutinin A (PHA), is assayed by the supplier (Invitrogen) and should be used at the recommended dilution.
All cultures are maintained in a water saturated atmosphere at 37 C. 5% CO₂.
Mononuclear cells and lymphocytes may also be collected using either (lymphocyte separation medium (Ficoll-Hypaque) or Lymphoprep tubes following the manufacturer's standard procedure.

When analyzed by fluorescent activated cell sorting, the regimen of IL-2 and PHA stimulation results in 99% CD3-positive cells (which stains all T cell subsets), with equal numbers of CD4-positive and CD4-negative cells (data not shown).

### C. Chaperone Treatment

The density of the T cells will be adjusted to 1×10⁶ per 3 ml of culture medium (RPMI-1640, 10% FBS, 25 ng/ml IL-2). 3 ml (˜1×10⁶ cells) will then be pipetted into each of 6 wells of a labeled 6-well culture plate and incubated overnight at 37° C. 5% CO₂. 3 ml of additional medium will then be added to 3 wells to give a final volume of 6 ml/well. To the three remaining wells, 3 ml of medium containing DNJ (Cambridge Major Laboratories. Inc., Germantown. WI) will be added at a concentration of about 40 μM (2×; final concentration is 20 μM), for 4-5 days. Cells will be harvested by centrifugation (400×g for about 10 minutes) and washed 1× in 10 ml PBS. The resulting pellets will be resuspended in 1 ml PBS and transferred to a 1.7 ml microfuge tube and centrifuged in a refrigerated microfuge at 3000 rpm for 5 minutes. The supernatant was aspirated and the pellets were stored frozen at −80° C. until assayed for enzyme activity.

Note that prior to conducting the enhancement assay, the optimum concentration of DNJ will be determined using a range from 2 nM-200 μM. For example, it may be determined that 20 μM is optimal.

### D. Activity Assay

Prior to assay, the T cells will be thawed on ice and sonicated for 2 minutes, and all other assay reagents will be thawed at room temperature. Fluorometric assay of GAA activity will be performed as follows. The cells will be lysed in 0.2 ml deionized water combined with vigorous pipetting and vortexing. The supernatant obtained after centrifugation at 13000 rpm for 2 min at 4° C. will be put into a fresh tube and used as the source of GAA. GAA activity will be determined by incubating 50 μl aliquots of the supernatant (containing comparable quantities of protein as determined using 20 μl in a standard protein quantitation assay) in a 24-well microplate at 37° C. with 3.75 mM 4-methyl umbeliferryl-α-D-glucopyranoside (4MU-alphaGlc) (Research Products International. Mount Prospect. Ill.) in the citric acid/phosphate buffer (27 mM citrate/46 mM phosphate buffer pH 4.6) without taurocholate and with BSA (3 mg/ml). A Wallac 1420 Victor3™ Fluorescence detection reader (Perkin Elmer, Calif.) will be used to measure the released 4-MU at excitation and emission wavelengths of 355 nm and 460 μm, respectively. Appropriate wells for fluorescent standards, and negative (no substrate or no lysate) will also be employed. For each patient sample at least three normal samples will be tested concurrently.

Incubations will typically be 30 minutes in duration but longer or shorter periods may be employed with similar results.

Enzyme activity (nmol/hr/mg of protein) will be calculated according to the following:

$$\frac{Fluorescence of sample}{Fluorescence of Standard} * \frac{60 mins}{Incubation time ( mins )} * \frac{1000 µL}{Volume assayed ( µL )} * \frac{1}{Protein value ( mg / mL )}$$

One unit of enzyme activity is defined as the amount of enzyme that catalyzes the hydrolysis of 1 nmole of 4-methyl umbeliferryl-α-D-glucopyranoside per hour. The baseline “noise” in the fluorescence output will be obtained by evaluating the average of blank six times. If the activity following SPC treatment is at least 2 standard deviations above the baseline, it will be considered responsive and not noise.

### Discussion

The use of T cells in a test system for enhancement of enzymes by SPCs offers significant advantages in the speed of assay and convenience over other culture systems. A critical step in determining which patients may benefit from SPC therapy is the development of a rapid and reliable method for screening of patient-derived cells for enhancement of GAA activity by DNJ. The results will demonstrate a method for quickly generating a short-lived cell culture that permits the testing of the enhancement and also provides a useful system for future studies on the mechanism of action or for screening of additional chaperone molecules. Leukocytes traditionally used for the diagnosis of affected status do not survive long enough to permit repeat assays if necessary.

Although Epstein-Barr virus transformed B lymphoblasts (Fan et al. Nat. Med. 1999: 5(1), 112-115) and primary fibroblast cultures (Fan, supra; Mayes et al. Clin Chim Acta. 1981; 112(2), 247-251) have been tested (see Example 1), a leukocyte test system provides for an additional, quick assay that may be easily used on a large scale for screening of patients for clinical studies.

The present invention provides a method for establishing T cell cultures from fresh blood of normal control individuals and patients with Pompe disease. These cultures can be grown for use in an enhancement assay for GAA in 7 to 10 days. It is expected that the effectiveness of DNJ enhancement will be evident after about 3 days in the T cell growth media. The data generated will be a reproducible measure of the degree of enhanced enzyme activity by a SPC for a specific genotype.

As with the lymphoblast test system, this method will be used for other SPC-based enhancement assays of other genetic diseases including glycosphingolipidoses and mucopolysaccharidoses, and can be extended as a research and clinical protocol in a wide range of genetically defined diseases, such as Cystic Fibrosis (CFTR) and cancer (p53, PTEN), among others.

### Prophetic Example 3

### In Vivo Method for Evaluating Effects of an SPC on GAA Activity

This example describes an open label Phase II study of DNJ in Pompe patients with different GAA mutations and will support the use of the in vivo assay. The patients will be selected for the Phase II study based on the increase in GAA activity in the lymphoblasr or T-cell assays described above.

Patients will be administered DNJ according to the dosing schedule described in U.S. Provisional Application 61/028,105, filed Feb. 12, 2008, herein incorporated by reference in its entirety. Blood will be draw into an 8 mL Vacutainer CPT tube at the end of each dosing period and treated as described below.

### A. Preparation of Human WBC Pellets for Assay

WBCs will be prepared substantially as described in Example 2, with the exception that no FBS/DMSO is added to the pellet prior to freezing.

### B. Preparation of Human WBC Lysates for Assay

To the microtubes containing the WBC pellet, 0.6 ml of lysis buffer (26 mM citrate/46 mM phosphate, pH 5.5) will be added
Tubes will be vortexed until the cells are re-suspended
Tubes will be incubated at room temperature for about 15 minutes, with agitation by vortexing every couple of minutes
Tubes will be sonicated for 2 minutes, then vortexed for about 10 seconds
Lysates will be incubated on ice until chilled, and then pooled into a pre-chilled polyproylene container (on ice)
Container will be vortexed, and pooled lysates will be divided into 0.100 mL aliquots in pre-chilled labeled 0.5 mL screw-cap polypropylene microcentrifuge tubes. Pooled lysates will be mixed while aliquoting by vortexing between every 10-20 aliquots.
Aliquots will be stored at −80° C. until use.

### C. Human WBC Assay

Each tube containing lysate will be thawed on ice, sonicated for 2 minutes, then vortexed for 1 minute.
50 μl of each standard, control, or clinical sample will be added into appropriate wells of a black polystyrene microplate (use 50 μl of 0.5% BSA in WBC lysis buffer for a standard)
50 μl of 5 mM 4MV-alphaGlc substrate will be added to all wells and the wells will be mixed on a plate shaker for 30 seconds
The plate will be covered and incubated for about 1 hour at 37° C.
100 μl of 0.2M NaOH/Glycine buffer, pH 10.7 will be added to each well to stop the reaction
The plate will be read using a fluorescent plate reader as described in Example 2

### Example 4

### Method to Measure GAA Enzyme in Muscle Tissue Homogenates

This Example describes how to measure acid α-glucosidase (GAA) enzyme activity in muscle biopsies. More specifically, during clinical trials, this method can be used to obtain necessary information on the pharmacodynamic effects of the investigational compound I-deoxynojirimycin (DNJ) on GAA in the target muscle tissues. The method was developed to reliably measure GAA activity in muscles that overcomes the potential problems of enzyme inhibition due to residual DNJ. This method relies on a lectin (concanavalin A)-bound matrix to capture GAA and other glycoproteins which enables efficient washing of the DNJ inhibitor prior to measuring GAA enzyme activity. This method can be used to better understand and develop effective dosing regimes for DNJ to increase GAA levels in Pompe patients.

### A. Reagents and Supplies

Bis-TRIS, Sigma B-9754
Glacial Acetic Acid, Sigma (99.7%)
Potassium Hydroxide
Sodium Carbonate, Sigma S-7795
Sodium chloride (5M), Promega V4221
Triton X-100, Sigma T-9284
Complete: Mini (EDTA-free) protease inhibitor cocktail tablets. Roche catalog#04 693 132 001
4-methyl-umbelliferyl-alpha-D-glucopyranoside (4-MU-α-D-glu) Sigma M-9766 (FW 338.31)
4-Methylumbelliferone (free dye), Sigma M-1381
Concanavalin A-Sepaharose 4B, Amersham Biosciences catalog#17-0440-01
Powermax tissue homogenizer AHS 200 (Pro Scientific, Thorofare, N.J.) VWR catalog#14227-318
Double deionized water
96-well plate, black plate with clear bottom, Costar 3603
BCA Protein Assay Kit, Pierce catalog#23225
Bovine Serum Albumin Standard, Pierce catalog#23209
multi-channel pipettors & tips
Single-channel pipettors & tips
Refrigerated microcentrifuge (e.g.
37° C. incubator
96-well fluorescence plate reader such as Victor3 (Perkin Elmer) or SpectraMax M2 (Molecular Devices)

### B. Solutions and Reagents

Stock 500 mM Bis-TRIS Buffer, pH 6.5
   Weigh out 104.62 g of Bis-TRIS in a clean 1 L beaker and dissolve in 800 ml ddH2O with stirring at room temperature.
   Adjust the pH of Bis-TRIS to 6.5 with HCl and add ddH2O to 1 L.
   Filter buffer through a bottle-cap filtering device equipped with a 0.2 μm membrane and store buffer at room temperature.
25× Protease inhibitor Solution
   Dissolve 1 tablet in 4 mL ddH2O per the manufacturer's instructions and store in 200 μl, aliquots at −80° C.
Bis-TRIS Buffer (25 mM Bis-TRIS-HCl/150 mM NaCl, pH 6.5)
   Add 25 mL of Stock 500 mM Bis-TRIS Buffer+15 mL of 5M NaCl Add H2O to a total of 500 mL.
   Filter buffer through a bottle-cap filtering device equipped with a 0.2 μm membrane and store buffer at room temperature.
Lysis Buffer (Bis-TRIS Buffer/1% (v/v) Triton X-100, protease inhibitor cocktail, pH 6.5)
   Add 0.5 mL Triton X-100 to 50 mL of Bis-TRIS Buffer for working stock solution.
   Note: Prepare Lysis Buffer immediately prior to use: Add 200 μL of 25× Protease Inhibitor Solution to 5 mL of Bis-TRIS Buffer/1% Triton X-100
   Place Lysis Buffer on ice until use
Pre-equilibrated ConcanavalinA-Sepahrose Resin
   Invert Concanavalin A (ConA)-Sepharose resin repeatedly (10-15 times) until slurry is a uniform mixture
   Transfer 6 mL of ConA-Sepharose slurry to a clean 15-mL centrifuge tube and spin down ConA-sepharose resin at 1000×g
   Determine amount of resin and discard storage buffer
   Wash resin by adding 2 volumes of Bis-TRIS Buffer and spin down resin at 1000×g; repeat washing procedure 2 additional times
   Add equal volume of Bis-TRIS Buffer to generate a 50% ConA-Sepharose slurry
   Use pre-equilibrated ConA resin for capturing GAA prior to enzyme activity assays
Stock KOAc Buffer (500 mM KOAc, pH 4.0)
   Add 28.8 mL glacial acetic acid (17.4 M stock) to 750 mL ddH2O
   Adjust pH to 4.0 with KOH and add ddH2O to 1 L
   Filter buffer through a bottle-cap filtering device equipped with a 0.2 μm membrane and store buffer at room temperature.
GAA Activity Assay Buffer
   Dilute 100 mL of Stock KOAc Buffer with 900 mL ddH2O
   Check pH to ensure that pH is 4.0
   Filter buffer through a bottle-cap filtering device equipped with a 0.2 μm membrane and store buffer at room temperature.
6 mM 4-MU-α-D-glucopyranoside in GAA Assay Buffer
   Allow vial to warm to room temperature.
   Weigh out 13.4 mg substrate in a clean 1.5 mL microcentrifuge tube
   Dissolve substrate in 200 μL 100% DMSO with brief vortexing
   Dilute substrate with 9.8 mL GAA Assay Buffer in a 15-ml conical tube. Store in the dark until use.
Free 4-MU Standards (5-30,000 nM corresponding 5e-13 to 3e-9 moles)
   Allow vial to warm to room temperature and weigh out approximately 5 mg of free 4-MU dye in a clean 1.5 mL microcentrifuge tube
   Dissolve the dye in an appropriate volume of 50% DMSO to obtain a 2.5 mM stock solution
   Aliquot (20 μL) and store the 4-MU stock in the dark at −80° C. until use *Note: Prepare 4-MU standards immediately prior to GAA enzyme activity assay
Thaw free 4-MU stock solution at room temp and vortex briefly
   Add 9.6 μL of 2.5 mM free 4-MU stock+190.4 μL Lysis Buffer for the 30,000 nM standard
   Perform serial dilution to obtain set of standards (0, 5, 50, 500, 5000, 15000 and 30000 nM)
400 mM Sodium Carbonate (pH˜11.5)
   Weigh out 21.2 g of Na2CO3 in a clean 500 mL beaker.
   Dissolve in 400 mL ddH2O with stirring at room temperature and add ddH2O to 500 mL
   Filter buffer through a bottle-cap filtering device equipped with a 0.2 μm membrane and store buffer at room temperature.

### C. Procedure

### 1. Tissue Homogenization

1. Weigh muscle biopsy sample in a clean 1.5 mL microcentrifuge tube
2. Add 200 μl of Pompe Lysis buffer per 50 mg muscle tissue (human biopsy samples)
Note: Add 500 μl of Lysis Buffer for normal muscle tissues
   3. Homogenize tissue on ice by repeated pulsing (3-5 times, 5 sec each pulse) using a micro-homogenizer (Pro Scientific)
Note: samples should be cooled in ice during the pulsing intervals so that samples do not get heated; extreme care should also be taken to avoid forming air bubbles during homogenization.
   4. Spin down debris by centrifugation at 9,200×g for 10′ at 4° C. and transfer supernatant to fresh 1.5 ml microcentrifuge tube.
   5. Use the supernatant for all downstream assays

### II. Determination and Adjustment of Protein Concentration

1. Aliquot 5 μL of each homogenate to a new microcentrifuge tube and dilute sample 1:10 (v/v) with Lysis Buffer
2. Use 10 μL of each diluted sample (in triplicate) to determine the total protein concentration using a BCA assay or similar method according to the manufacturer's instructions
3. If desired, adjust all samples to a common protein concentration (e.g. 5 mg/mL) with Lysis Buffer

### III. Concanavalin A (Con A) Capture and GAA Enzyme Activity Assay

1. Prepare samples in 1.5 mL, microcentrifuge tubes by adding 50 μL pre-equilibrated ConA-Sepharose resin (50% slurry)
2. Add 100 μg of total protein from each tissue homogenate
3. Add Bis-TRIS Buffer to tubes such that the final volume is 500 μL for all samples
4. Incubate samples at room temp for 30 minutes with rocking
5. Spin down ConA-Sepharose at 5000×g for 10-15 seconds and carefully remove the supernatant without disturbing the resin
6. Wash ConA resin by adding 500 μL of Bis-TRIS Buffer, inverting tubes 5 times, spin down at 5000×g for 10-15 seconds and discard supernatant
7. Repeat steps 5 and 6 two additional times and remove supernatant from final wash
8. Add 100 μL of GAA Activity Assay Buffer to each microcentrifuge tube
9. Mix Con A resin by repeated pippetting (˜10 times) using large-bore tips and transfer 20 μL of slurry of each sample to a black 96-well assay plate (perform triplicate for each sample)
10. Add 50 μL of 6 mM 4-MU-α-D-glucopyranoside substrate solution to all wells EXCEPT free 4-MU standards wells
11. Add 4-MU standards in designated wells
12. Incubate plate at 37° C. for 2 hours
13. Stop reaction by adding 70 μL of 400 mM Sodium Carbonate Buffer to all wells
14. Read in a fluorescence plate reader (370 nm excitation/460 nm emission)
15. Extrapolate GAA activity from 4-MU standard curve and report activity as nmol 4-MU released/mg total protein/hr

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purpose.

What is claimed is:

1. A method for determining whether a patient having a deficiency in activity of a protein will respond to treatment with a specific pharmacological chaperone for the protein, which method comprises
a. contacting cells in or from a patient with a specific pharmacological chaperone (SPC) for the protein; and
b. comparing protein activity in cells not contacted with specific pharmacological chaperone, with protein activity in cells contacted with the specific pharmacological chaperone
wherein a sufficient increase in protein activity in cells contacted with the specific pharmacological chaperone (SPC) over activity in cells not contacted with the specific pharmacological chaperone (SPC) indicates that the individual will respond to treatment with the specific pharmacological chaperone (SPC).

2. The method of claim 1, wherein the deficiency of activity is caused by a missense mutation in a gene encoding the protein.

3. The method of claim 1, wherein the protein is an enzyme.

4. The method of claim 3, wherein the enzyme is a lysosomal enzyme.

5. The method of claim 4, wherein the patient has been diagnosed with a lysosomal storage disorder.

6. The method of claim 5, wherein the lysosomal enzyme is α-glucosidase and the lysosomal storage disorder is Pompe disease.

7. The method of claim 5, wherein the specific pharmacological chaperone is 1-deoxynojirimycin and said cells are selected from the group consisting of white blood cells, lymphoblasts and fibroblasts

8. The method of claim 7, wherein the cells are white blood cells and the contact with the specific pharmacological chaperone occurs ex vivo.

9. The method of claim 7, wherein the cells are T lymphocytes and the contact with the specific pharmacological chaperone occurs in vitro.

10. The method of claim 9, wherein the T lymphocytes are obtained by
a. separating white blood cells from a blood sample obtained from the patient;
b. washing white blood cells; and
c. establishing a cell culture enriched with T lymphocytes.

11. The method of claim 10, wherein the T lymphocytes cultured in the absence or in the presence of the specific pharmacological chaperone 1-deoxynojirimycin for about 1-3 days.

12. The method of claim 11, wherein the culturing in the absence or presence of 1-deoxynojirimyicin is for about 3 days.

13. The method of claim 12, wherein α-glucosidase activity is determined using a fluorometric assay that quantities hydrolysis of substrate in lysates from the T lymphocytes.

14. The method of claim 12 wherein the sufficient increase in activity in the lysates in the presence of the 1-deoxynojirimycin which indicates whether the patient will respond is measured according to the following criteria:
i) If baseline activity is less than 1% of normal, the activity following culture or following treatment with SPC must be at least 2% of normal;
ii) If baseline activity is between 1% but less than 3% of normal then the activity following culture or treatment with SPC must be at least 2 times the baseline level;
iii) If baseline activity is between 3% but less than 10% of normal, then the activity following culture or treatment with SPC must be at least 3% of normal higher the baseline level of normal;
iv) If baseline activity is 10% of normal or more, then activity following culture or treatment with SPC must be at least 1.3× the baseline level.

15. The method of claim 12, wherein the sufficient increase in activity the presence of the 1-deoxynojirimycin which indicates whether the patient will respond is between about 2-fold and 25-fold over the activity in the absence of the 1-deoxynojirimycin.

16. The method of claim 15, wherein the sufficient increase in activity the presence of the 1-deoxynojirimycin which indicates whether the patient will respond is at least 20% over the activity in the cells not cultured with 1-deoxynojirimycin.

17. The method of claim 8, wherein the patient is administered 1-deoxynojirimycin daily for about 2 weeks.

18. The method of claim 17, wherein the administration is oral.

19. The method of claim 17, wherein the 1-deoxynojirimycin is administered at a dose of about 50-4000 mg/day.

20. The method of claim 19, wherein the dose is about 250-3000 mg/day.

21. The method of claim 20, wherein the dose is about 2500 mg/day.

22. The method of claim 19, wherein the 1-deoxynojirimycin is administered once a day.

23. The method of claim 17, further comprising collecting a blood sample at the end of two weeks and separating the white blood cells.

24. The method of claim 17 wherein GAA activity is determined using a fluorometric assay that quantifies hydrolysis of substrate in lysates from the white blood cells.

25. The method of claim 24 wherein the sufficient increase in activity in the lysates in the presence of the 1-deoxynojirimycin which indicates whether the patient will respond is measured according to the following criteria:
i) If baseline activity is less than 1% of normal, the activity following culture or following treatment with SPC must be at least 2% of normal;
ii) If baseline activity is between 1% but less than 5% of normal then the activity following culture or treatment with SPC must be at least 2 times the baseline level;
iii) If baseline activity is between 5% but less than 10% of normal then the activity following culture or treatment with SPC must be at least 5% of normal higher the baseline level of normal;
iv) If baseline activity is 10% of normal or more, then activity following culture or treatment with SPC must be at least 1.5× the baseline level.

26. The method of claim 7, wherein the white blood cells are T lymphocytes and the contact with the specific pharmacological chaperone occurs in vitro.

27. The method of claim 7, wherein the cells are lymphoblasts and the contact with the specific pharmacological chaperone occurs in vitro.

28. The method of claim 27, wherein the lymphoblasts are obtained by
a. separating white blood cells from a blood sample obtained from the patient;
b. washing white blood cells; and
c. establishing a lymphoblast cell line.

29. The method of claim 28, wherein the lymphoblasts cultured in the absence or in the presence of the specific pharmacological chaperone 1-deoxynojirimycin for about 1-5 days.

30. The method of claim 29, wherein the culturing in the absence or presence of 1-deoxynojirimyicin is for about 5 days.

31. The method of claim 30, wherein α-glucosidase activity is determined using a fluorometric assay that quantifies hydrolysis of substrate in lysates from the lymphoblasts.

32. The method of claim 30 wherein the sufficient increase in activity in the lysates in the presence of the 1-deoxynojirimycin which indicates whether the patient will respond is measured according to the following criteria:
i) If baseline activity is less than 1% of normal, the activity following culture or following treatment with SPC must be at least 2% of normal;
ii) If baseline activity is between 1% but less than 3% of normal then the activity following culture or treatment with SPC must be at least 2 times the baseline level;
iii) If baseline activity is between 3% but less than 10% of normal, then the activity following culture or treatment with SPC must be at least 3% of normal higher the baseline level of normal;
iv) If baseline activity is 10% of normal or more, then activity following culture or treatment with SPC must be at least 1.3× the baseline level.

33. The method of claim 30, wherein the sufficient increase in activity the presence of the 1-deoxynojirimycin which indicates whether the patient will respond is between about 2-fold and 700-fold over the activity in the absence of the 1-deoxynojirimycin.

34. The method of claim 33, wherein the sufficient increase in activity the presence of the 1-deoxynojirimycin which indicates whether the patient will respond is at least 20% over the activity in the cells not cultured with 1-deoxygnojirimycin.

35. A kit comprising:
a. at least one T cell stimulatory agent;
b. a specific pharmacological chaperone;
c. a labeled substrate for the chaperone; and
d. instructions for performing a protein enhancement assay.

36. The kit of claim 26, wherein the T-cell stimulatory agent is a mitogen.

37. The kit of claim 27, wherein the mitogen is PHA.

38. The kit of claim 26, wherein the stimulatory agent is a cytokine.

39. The kit of claim 29, wherein the cytokine is IL-2.

40. The kit of claim 26, wherein the pharmacological chaperone is 1-deoxynojirimycin.

41. The kit of claim 26, further comprising one or more a blood collection tubes, centrifuge tubes, and cryotubes.

42. The kit of claim 26, wherein the protein is an enzyme.

43. The kit of claim 33, wherein the enzyme is α-glucosidase.

44. A method for increase the sensitivity and accuracy of GAA activity measurement in DNJ treated patient tissue homogenate samples, which method comprises using a lectin (concanavalin A)-bound matrix to capture GAA and other glycoproteins and washing DNJ prior to measuring GAA enzyme activity.
